(19) United States
(12) Patent Application Publication
(10) Pub. No.: US 2005/0177201 A1
(43) Pub. Date: Aug. 11, 2005

(54) Probe insertion pain reduction method and device

(76) Inventor: Gary A. Freeman, Newton, MA (US)

Correspondence Address: FISH & RICHARDSON PC, 225 FRANKLIN ST, BOSTON, MA, 02110, US

(21) Appl. No.: 10/403,686

(22) Filed: Mar. 31, 2003

(51) Int. Cl. A61N 1/08; A61N 1/10

(52) U.S. Cl. 607/46; 607/117

(57) ABSTRACT

In general the invention features inserting a probe element through the skin by moving the probe element along a penetration path in a series of incremental movements. The incremental movements produce incremental penetrations of the skin that are each small enough not to produce substantial stimulation of nerve axons (e.g., nociceptor axons).

### BACKGROUND

This invention relates generally to a hypodermic needle, wire, trocar, catheter or other subcutaneous probe insertion method, and to a device utilizing such a method.

There are numerous ailments which require the insertion of a probe subcutaneously for treatment. Acupuncture requires the insertion of multiple fine wires. Application of a local anesthetic to block nerve transmission such as in oral surgery is often associated with significant pain accompanying the insertion of the hypodermic syringe prior to the anesthetic taking effect. Chronic diseases such as diabetes mellitus require as many as several daily subcutaneous injections of insulin to compensate for the body's inability to produce or utilize sufficient quantities of insulin. In addition, the diabetes mellitus patient must also test for their blood glucose levels as many as five times a day. The two primary goals of any glucose monitoring and insulin injection system are patient comfort and better glycemic control. Good glycemic control is directly related to reduced risk of complications in diabetes patients. Increased patient convenience and comfort have a direct, positive effect on the patient's treatment compliance, resulting in improved glycemic control and patient health. Continuous infusion pumps such as the MiniMed (Medtronic, Minneapolis) require a subcutaneous catheter or needle that is changed by the patient every two or three days.

The pain and discomfort of probe insertions of these types is an inhibition to full patient compliance and treatment. Cutaneous sensory receptors are typically categorized according to the type of stimulus to which they respond. Mechanoreceptors respond to mechanical stimuli such as stroking or indenting. Hair follicle receptors, Meissner's and Pacinian corpuscles, Merkel cell endings and Ruffini endings all fall under the category of mechanoreceptors. The second type of cutaneous sensory receptor, thermoreceptors, respond to the temperature of the skin. A third set of receptors, chemoreceptors respond to a variety of chemicals to provide the receptors for the senses of smell and taste. A fourth set of receptors, nociceptors, respond to stimuli that may be harmful by signaling pain. Two types of nociceptors are the delta-type A (Aδ) fibers and the C-polymodal fibers. The Aδ mechanical nociceptors respond to stimuli such as a needle prick; they do not respond to thermal or chemical stimuli. C-polymodal nociceptors, on the other hand, respond to noxious mechanical, thermal and chemical stimuli. When a receptor is stimulated, it produces a voltage level called a generator potential at the terminal end of its axial connection, and if the generator potential is of sufficient amplitude and duration, it will initiate a nerve impulse called an action potential (AP). The AP travels electrochemically along the fiber called the nerve axon. Nociceptors are afferent nerve cells, i.e. they carry information form the body's sensory system to the brain via the spinal cord.

The stimulation of cutaneous nociceptor nerve axons follow the standard strength-duration relationship describing the excitation of nerves as first derived by Weiss in 1901 and expressed in Lapicque's formula:
I_{T}=I₀[1−exp^{-(t/τe)}]⁻¹,
where I_{T} is the amount of current required to cause an AP.

Lapicque defined “rheobase” as the minimum activation current for long pulses (I₀ in the equation) and “chronaxie” as the duration of the threshold current having a magnitude of twice the rheobase (τₑln 2=τₑ×0.693 in the formula.) The intensity of the stimulus may be encoded by the sensory receptors by the mean frequency of discharge of sensory neurons. The generator potential, unlike the ‘all or nothing’ action potential, is graded and the AP repetition rate will be a function of the amplitude and duration of the generator potential. This relationship between the stimulus and response is typically plotted as a stimulus/response function, with the general form of the equation for such a function:
Response=K*(Stimulus−threshold stimulus)ⁿ,
where K is a constant and n is an exponent. More detailed models such as the Hodgkin-Huxley and Frankenhaeuser-Huxley model have been developed incorporating models for actual membrane ion flux and other relevant biophysical parameters. Stimulus-response functions for mechanoreceptors typically have fractional exponents, while thermoreceptors have exponents close to one (approximately linear functions). Nociceptors, often have exponents greater than one.

Stimulus intensity may also be encoded by the number of receptors activated. Stimuli of different intensities may also activate different sets of sensory receptors. For instance, a particular mechanical stimulus with a small amplitude may only activate mechanoreceptors, while the same stimulus of a larger amplitude might activate both mechanoreceptors and nociceptors.

Methods have been developed for minimizing the pain of probe insertion. U.S. Pat. No. 6,517,521 utilizes a needle with one or more perforations in its side to reduce the localized tissue distension caused by the fluid injection. The structure results in a broader distribution of the injected fluid. U.S. Pat. No. 5,681,283 seeks to reduce the sensation of pain by reducing the total duration via high velocity insertion. U.S. Pat. No. 5,236,419 teaches numbing the outer tissue layers by chilling prior to needle insertion. U.S. Pat. No. 6,501,976 describes a method where a microneedle is inserted just below the dermal or epidermal layers to avoid stimulating the nocicepteptors. Other methods have been developed that avoid the use of needles entirely: U.S. Pat. Nos. 5,879,367, 6,120,464, 5,019,034, 6,091,975 and 6,468,229 teach methods for sampling interstitial body fluids with minimal or no probe insertion. U.S. Pat. No. 5,501,666 employs a needleless system via a jet injection of fluids. Other methods include prior treatment of the injection area with local anesthetics either topically or subcutaneous injection. In the field of acupuncture, pre-treatment of the insertion area with electrical energy, often in the form of high-frequency waveforms typically used for transcutaneous electrical nerve stimulation (TENS), is employed to reduce the discomfort of insertion as well as provide optimal placement and treatment. U.S. Pat. Nos. 3,939,841, 5,385,150, 5,546,954, 6,516,226, 6,493,592, 6,516,226 and 6,522,927 employ variations of this technique. U.S. Pat. No. 4,363,326 combines an ultrasonic function with a needle probe, but the only purpose the ultrasonic function serves is as a means of imaging tissue beneath the probe, and the needle probe is separated from the ultrasonic transducers.

### SUMMARY

In general the invention features inserting a probe element through the skin by moving the probe element along a penetration path in a series of incremental movements. The incremental movements produce incremental penetrations of the skin that are each small enough not to produce substantial stimulation of nerve axons (e.g., nociceptor axons).

In preferred implementations, the invention may incorporate one or more of the features recited in the appended claims.

The invention has numerous advantages over the current art. Some of the advantages may only be achieved with some implementations of the invention.

The reduced pain of needle insertion may make modes of treatment such as acupuncture more appealing to patients and results in less patient discomfort when receiving hypodermic injections. There is a particular benefit to patients suffering from chronic diseases like diabetes mellitus which require piercing of the skin for blood glucose measurement and injection of insulin on a daily basis. Better glycemic control and improved long-term patient health can be achieved by making the task of glucose measurement and insulin injection less painful to the patient. In some implementations of the invention, the elements for moving the probe can be incorporated into a device that is compact enough to fit onto the proximal end of existing manual syringes without any modifications to the syringe barrel. Reducing the pain of hypodermic injections in pediatric medicine is desirable.

When any probe is inserted through a puncture resistant tissue such as skin or other membrane into a softer underlying tissue, the puncture resistant layer will naturally compress. When the puncture of the membrane occurs, the probe will extend to approximately the compression depth into the underlying tissue. This may result in a greater penetration depth than intended, with resulting damage to the underlying tissue. In conventional hypodermic injections of vaccines this may not be an issue. There is, however, a need to insert medical electrodes into nerve tissue such as the cerebral cortex, brain stem and spinal cord, and to be able to accurately control the insertion depth. The electrode must penetrate the puncture resistant pia-arachnoid member overlapping the cortex and spinal cord, but then once that layer has been pierced, the electrode's position must be quickly stabilized to prevent injury to the underlying neural population and vasculature. Some implementations of the invention provide such accurate control of penetration depth.

Prior art such as U.S. Pat. No. 6,304,785 teach a viscous-damped insertion mechanism that has an initially high insertion velocity which facilitates the piercing of the pia-arachnoid member, followed by a deceleration to aid in stabilizing the electrode position in order to accommodate the initial compression of the outer membrane. In some implementations of the invention, the probe is in constant oscillatory motion, with resulting reduction in insertion friction and stiction (the nonlinear force present at the onset of motion), and thus significantly less compression of the outer membrane. The actual insertion velocity (as measured by the distance between the probe proximal end and the desired final probe location such as adjacent to specific nerve tissue) may be maintained at a more constant rate thus reducing the potential for tissue damage.

Noninvasive glucose measurement technologies don't provide a means of insulin injection, which must be accomplished via a separate injection by the patient. The ideal system for glycemic control would have both glucose measurement and infusion in a system that is comfortable and convenient for the patient. Some implementations of the invention would allow for such a system. Currently available commercial continuous insulin pumps still need to have catheters replaced every 2 or 3 days. The catheter replacement is a painful procedure for the patient. Some implementations of the invention could be incorporated into continuous pump systems to reduce the pain of catheter insertion.

Other features and advantages of the invention will be apparent from the drawing, detailed description, and claims.

### BRIEF DESCRIPTION OF THE DRAWINGS

FIGS. 1a-1e are plots illustrating movement of the probe element.

FIGS. 2a-2c are plots illustrating movement of the probe element for the case of a sawtooth waveform.

FIG. 3 shows a biphasic waveform for the penetration curve.

FIG. 4 shows a randomized amplitude waveform for the penetration curve.

FIG. 5 shows a manual hypodermic syringe with a probe insertion device attached to its proximal end.

FIG. 6 shows a block diagram of the preferred embodiment of the device.

FIG. 7 shows a cross-sectional view of motor unit of FIG. 5.

FIG. 8 shows an implementation of the needle assembly of a magnetic actuator of the device in FIG. 5.

FIG. 9 shows an implementation of a piezoelectric actuator of the device in FIG. 5.

FIG. 10 shows a continuous injection insulin pump as worn on a patient's arm.

FIG. 11 shows the block diagram for the device of FIG. 10.

FIG. 12 shows a cross-sectional view of the housing of the device shown in FIG. 10.

FIG. 13 shows a detail of the disposable cartridge containing the insulin reservoir and needle as used in the device of FIG. 10.

FIG. 14 shows a catheter-type probe with a needle used as a puncture element.

FIG. 15 shows a cross-sectional view of the probe of FIG. 14 with the needle exposed to show the glucose sensing element.

FIG. 16 shows a finger probe for glucose measurement.

### DETAILED DESCRIPTION

There are a great many possible implementations of the invention, too many to describe herein. Some possible implementations that are presently preferred are described below. It cannot be emphasized too strongly, however, that these are descriptions of implementations of the invention, and not descriptions of the invention, which is not limited to the detailed implementations described in this section but is described in broader terms in the claims.

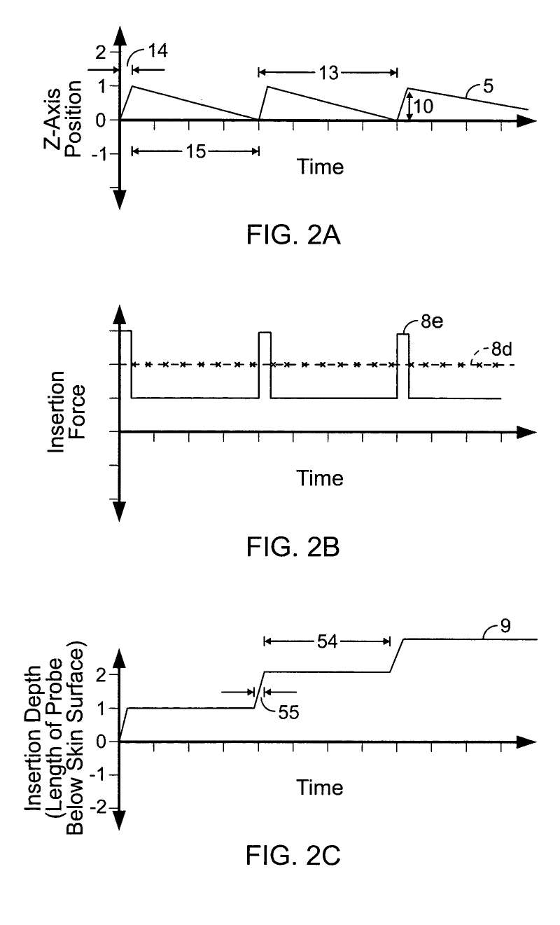

One implementation of the invention is described in FIG. 2. The probe element 1 is actuated in an incremental motion substantially along the probe axis by means of a motor element 2, with the amplitude of the incremental motion being less than the overall insertion depth. The operation of the motor element 2 is controlled by a motor control element 3 and powered by a motor power element 4. By inserting the probe element with incremental penetrations, the stimulation of nociceptors can be reduced or eliminated.

Although the invention is not limited to any theory for the pain reduction achieved, we believe that the mechanism for the reduction in nociceptor stimulation is as follows:

The probe insertion device moves the probe element along a penetration path in a series of incremental movements which produce incremental penetrations of the skin. Each penetration is substantially smaller than the penetration depth and also small enough not to produce substantial stimulation of nerve axons associated with nerve receptors located along the penetration path. Additionally, the incremental penetrations are spaced apart in time to reduce stimulation of neurons along the penetration path as well any neurologic integrative effects that might occur as a result of multiple stimuli. A more detailed theoretical description follows.

As was previously mentioned, cutaneous sensory receptors are typically categorized according to the type of stimulus to which they respond. Nociceptors respond to stimuli that may be harmful by signaling pain. The stimulation of cutaneous nociceptor nerve axons follow the standard strength-duration relationship describing the excitation of nerves. Repetitive stimuli can be more potent than a single stimulus as a result of threshold reduction or response enhancement; in both cases there is an integrative effect that acts to sum, to a greater or lesser extent, the multiple stimuli.

Threshold reduction occurs at the membrane level of the nerve cell. When stimulating the nerve axon to multiple generator potential pulses, the membrane integrates the pulse over a duration on the order of the membrane time constant, τₑ. In studies reported by J. P. Reilly et al, it was found that, in the case of a 20 μm myelinated nerve fiber, for monophasic pulses spaced further apart than 500 μs, there was no integrative effect. This is approximately 4 times the time constant for the fiber. As the number of pulses was increased from two to thirty-two in the stimuli pulse train, additive thresholds reached a minimum at 4-8 pulses in all cases. In the case of sinusoidal waveform stimulation, Reilly exposed the nerve to varying numbers of sinusoidal cycles and determined the threshold. Additive thresholds reached a minimum at 8 cycles for 5 kHz, 64 cycles for 50 kHz, and no decrease in the case of 500 Hz; in each of the three cases, the integrative time period is approximately 2 ms. Threshold reduction may also occur on a longer time scale, on the order of 1 second and longer, as a result of hyperalgesia, a process of sensitization of nociceptors. Sensitization occurs when chemical products released as a result of inflammation or cell damage reduce the nociceptor thresholds in the region of the chemicals.

Response enhancement occurs at higher levels within the central nervous system for neurosensory effects. Researchers have reported results for electrical stimulation of pain (5 ms pulse width with a period of 10 ms) that showed a 50% threshold reduction after ten pulses.

The stimulus response function of nociceptors are non-linear in two respects: 1) as previously stated, the exponents in their stimulus-response functions are greater than one; 2) the activation threshold for nociceptors is higher than that of mechanoreceptors so that a particular mechanical stimulus with a small amplitude may only activate mechanoreceptors, while the same stimulus of a larger amplitude might activate both mechanoreceptors and nociceptors.

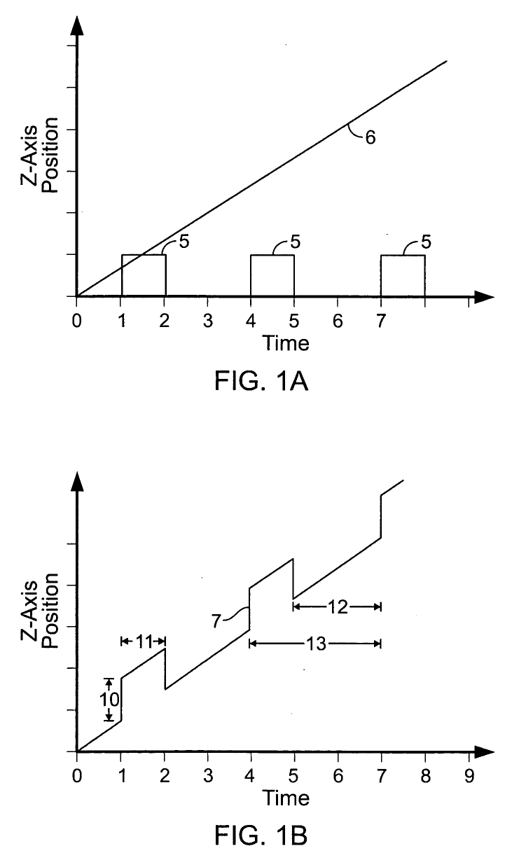

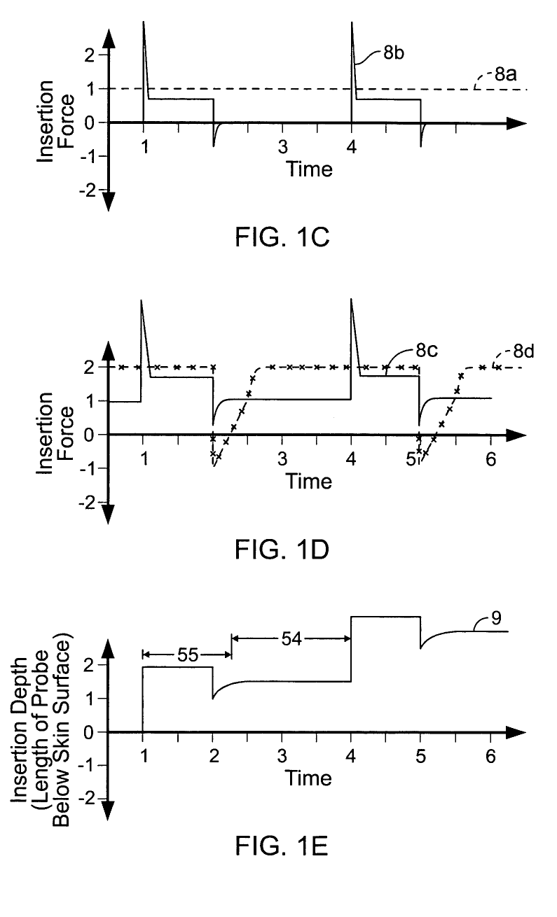

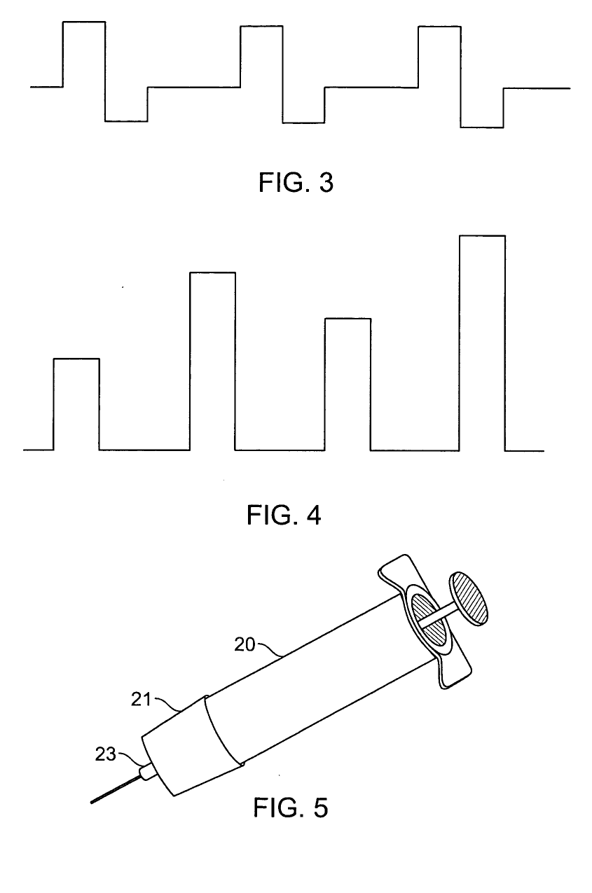

Based on the non-linear stimulus response function, the strength duration relationship of nociceptor membrane stimulation, and the threshold reduction effects of multiple pulses, at least some implementations of the invention operate on the principle of cutaneous penetration via subthreshold nociceptor stimulation. In one implementation, during the course of probe insertion, the proximal end of the probe is advanced relative to the membrane in small increments relative to the overall desired insertion depth. As shown in FIGS. 1a and 1b, the position of the proximal end of the probe may be viewed as the superposition of two motions, the penetration curve 5 and the insertion curve 6, resulting in the total z-axis position curve 7. It should be noted that because of the compliance of the skin, the insertion depth 9, as measured by the length of the probe below the skin's surface, will be different than the total Z-Axis position curve, as shown in FIG 1e. FIGS. 1c-1e show the insertion process from the perspective of the various forces in the system. The penetration force 8b is the result of penetration curve 5. The insertion force 8a is the result of insertion curve 6 and is less than the pain threshold of the patient. The total insertion force 8c is the result of the superposition of insertion force 8a and the penetration force 8b. The penetration threshold 8d is the force required for the probe to proceed further into the skin. At times 1 and 4 (arbitrary units), the total insertion force 8c exceeds the penetration threshold 8d and the insertion depth 9 increases. On the return stroke of the probe, the needle will partially retract from the opening, but because a cavity has been created beneath the probe tip and the skin is under compression, the penetration threshold 8d will decreases. Within a short period of time the compressed tissue will push the probe back into the cavity it just created. The individual pulse width, W_{P} 11, and pulse amplitude, A_{P} 10, of the penetration curve 5 are set so as to provide subthreshold stimuli to nociceptors in the region of insertion. The pulse period, τ_{P} 13, is set to provide a sufficient period of time between pulses, δ_{P} 12, so as to minimize the integration effects of multiple pulses. For monophasic rectilinear pulses, W_{P} 11 is typically set in the range of 10 μs-10 ms, though preferably it is in the range of 100-500 μs. τ_{P} 13 is set to 100 μs-500 ms, though preferably in the range of 100 μs-10 ms. The slopes of the rising (insertion) edge 14 and the falling (removal) edge 15 of the pulse can be adjusted so as to stimulate different groups of receptors. In one implementation, the rising edge 14 is preferably less than 1 ms and the falling edge 15 is greater than 1 ms and preferably greater than 40% of δ_{P} 12 resulting in a sawtooth-type waveform for the z-position curve 5 as shown in FIG. 2. A_{P} 10 is set to 1 μm-1 mm, though preferably to 5-100 μm. The amplitude is typically dependent on both the rising edge 14 slope (insertion velocity) and τ_{P} 13. The skin is most sensitive to vibratory stimulus at around 300 Hz, being able to detect displacement of approximately 1 μm. That sensitivity decreases logarithmically to 32 μm at 30 Hz and 1 mm at 3 Hz. In the case of the sawtooth waveform, if the falling edge slope (retraction velocity) 15 is linear and less than the response time of the compressed tissue, the total insertion force 8e will be substantially flat with pulses occurring with the rising edge 14 slope of the sawtooth. In this case, the insertion depth curve 9 will approximately take the shape of a staircase, which is optimal for sensation minimization. The term Insertion Pulse Spacing 54 is hereinafter used in this disclosure to mean the substantially constant portions of the insertion depth curve in between insertion pulses, as illustrated in FIGS. 1e and 2c, during which there is little or no nociceptor stimulation. In the case of the monophasic waveform, the Insertion Pulse Spacing 54 corresponds to δP 12 of the position waveform, and in the case of the sawtooth waveform, the Insertion Pulse Spacing 54 corresponds to the falling edge 15. The term Insertion Pulse Width 55 is used herein to refer to duration of time between the insertion and (if present) removal times of the insertion pulse as shown in FIGS. 1e and 2c. It should be noted that in the case of the sawtooth waveform, where there is essentially no removal portion of the insertion depth curve, Insertion Pulse Width 55 corresponds to only the rising (insertion) edge 14. The waveform may take a variety of shapes, among them a biphasic as shown in FIG. 3 or a waveform with randomized pulse amplitudes as shown in FIG. 4.

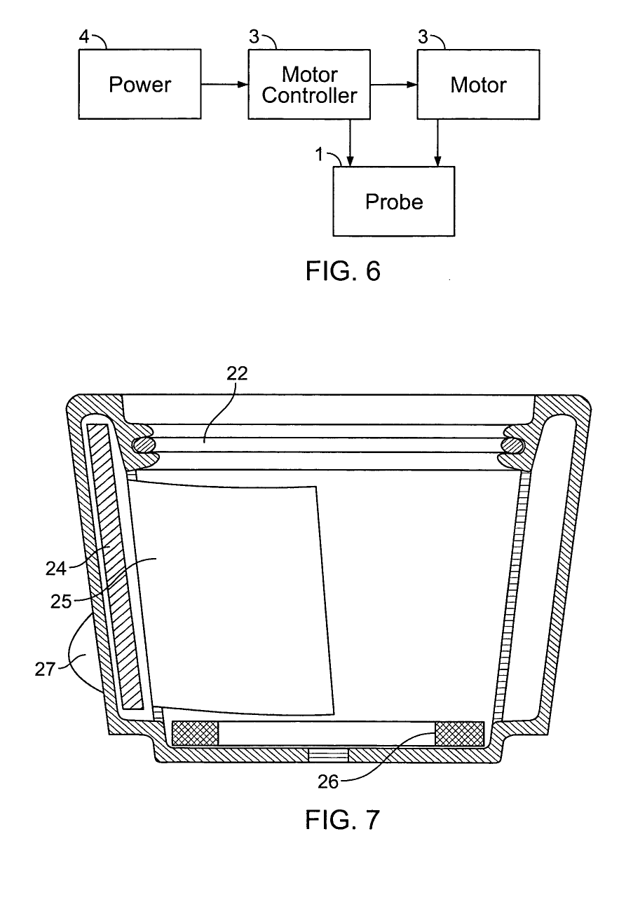

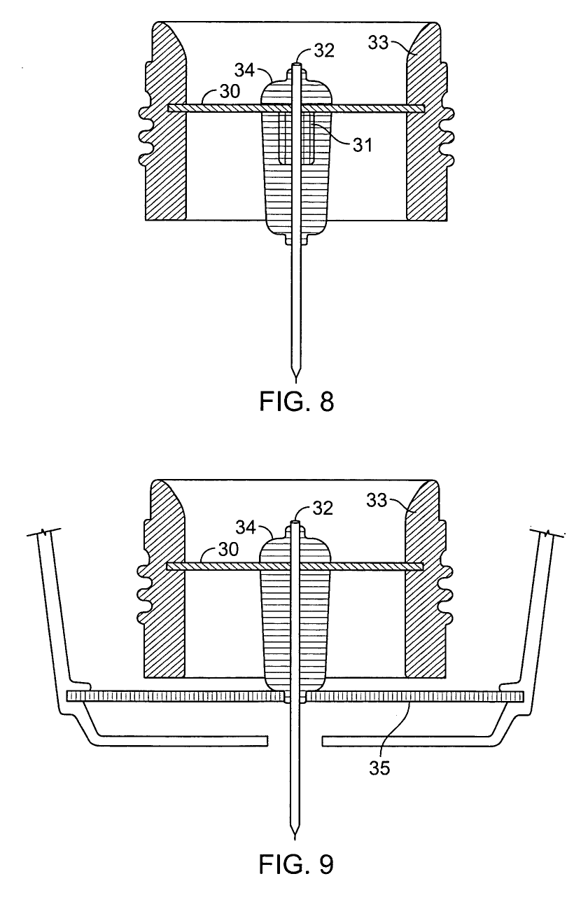

In one implementation, the device incorporating this above-mentioned probe insertion method is configured as a device that can be attached to existing manual hypodermic syringes as shown in FIG. 5-8. The syringe barrel 20 is inserted into the motor unit 21 and is held in place via an o-ring 22 providing a compression fit. The needle assembly 23 is affixed to the syringe barrel's existing needle mount. A block diagram for the motor unit 21 and needle assembly 23 is provided in FIG. 6. In the preferred configuration, the motor uses magnetic actuation with the actuator coil 26 enclosed in the motor unit 21. The magnet 31 for magnetic actuation is contained in the needle assembly 23 as shown in the cross-sectional view FIG. 8. A flexible diaphragm 30 is inserted on the needle shaft 32 above the magnet 32. The magnet 31 and flexible diaphragm 30 are affixed to the needle shaft with a small overmolded polymer shell 34. The thread mount barrel 33 is overmolded onto the outer edge of the diaphragm 30. The thread mount 33 provides the means of affixing the needle assembly 23 to the syringe barrel 20. A cross-sectional view of the motor unit is provided in FIG. 7. The electromagnetic coil 26 is located at the base of the motor unit 21 in alignment with the magnet 31 of the needle assembly 23 to provide maximum magnetic field transmission between coil and magnet. Electronic circuitry for the motor controller 3 (as shown in FIG. 6) is contained on the flexible circuit 25 using standard polyimide or polyester based flexible electronic substrates. Power 4 (as shown in FIG. 6) is preferably provided by battery 24. The battery is preferable a rechargeable secondary battery, preferable a lithium ion type. Charging is accomplished through use of the coil 26 and a separate base charger unit by means of magnetic induction. Power may also be provided by a primary battery, fuel cell, spring-powered mechanical generator or other means. An On/Off switch 27 is provided on the side of the motor unit 21. When the unit is turned on, the needle shaft 32 travels in a substantially vertical motion as described in this section by means of the force induced on the magnet 31 from the coil's magnetic field.

In an alternative implementation, the actuator may be a piezoelectric actuator, as shown in FIG. 9. FIG. 9B shows a cross-sectional view of the needle assembly 23 modified to accommodate a piezoelectric actuator. The piezoelectric element 35 replaces the coil 26 in the motor unit 21. The piezoelectric element 35 is in the shape of a disk, with features on the proximal end of the overmolded polymer shell 34 seated in a central hole in the piezoelectric element 35. By dimensioning the overmolded polymer shell 34 properly, the diaphragm 30 may be held in a stretched position when the motor unit 21 is attached. This is particularly helpful for the implementation where the penetration curve 5 takes the form of a sawtooth waveform. In this case, during the insertion edge 14, the mass that the piezoelectric actuator is driving is only that of the actuator itself, while on the removal edge 15, the mass is increased by the needle and diaphragm, along with the opposing force of the diaphragm itself. This ‘variable mass’ configuration allows for substantially increased insertion velocities.

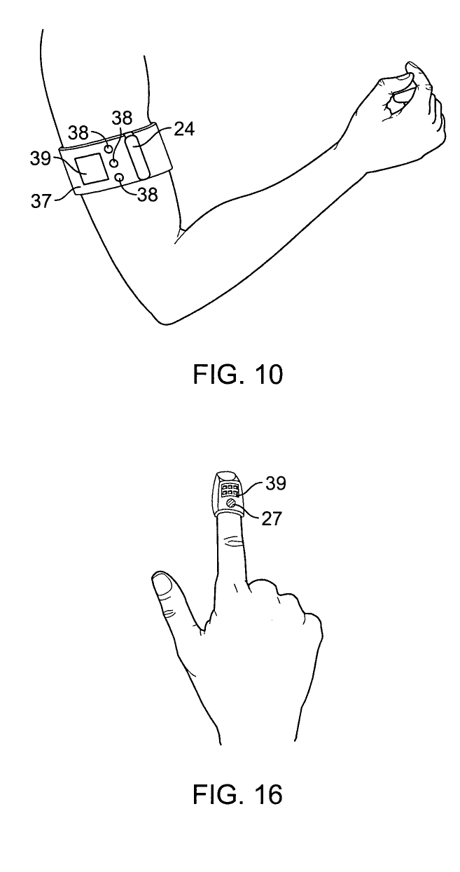

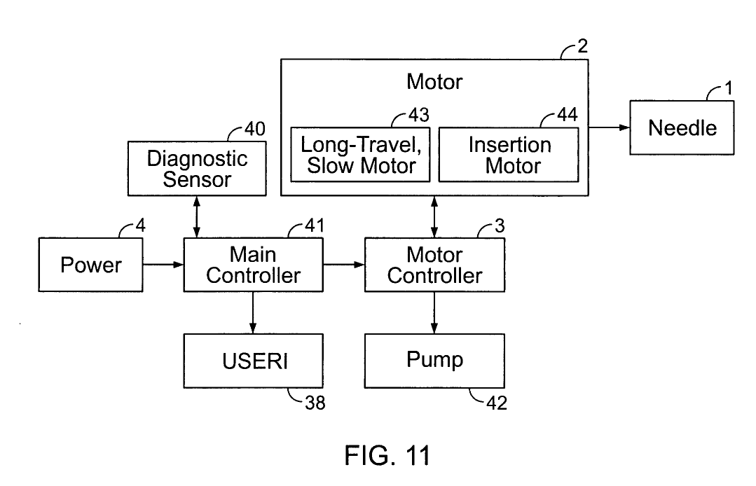

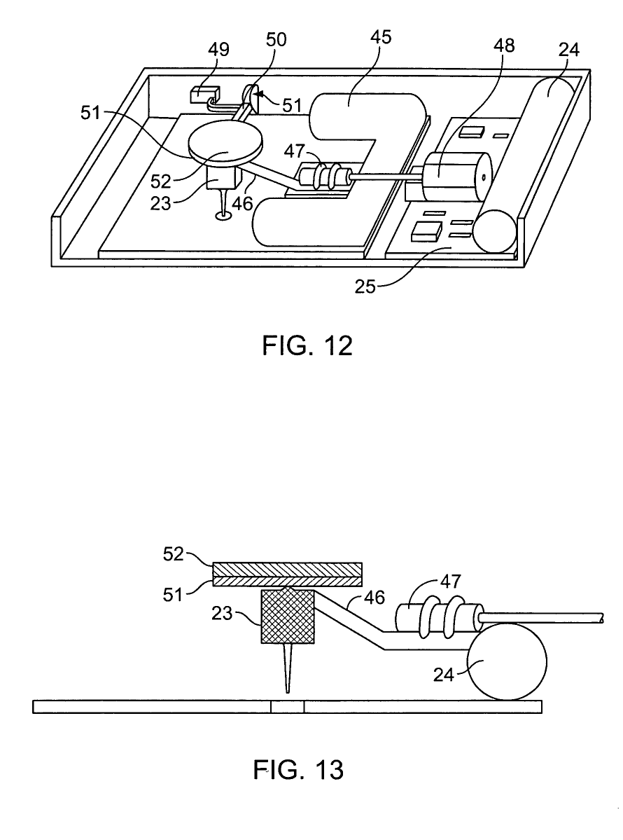

In another implementation, the device incorporating this above-mentioned probe insertion method is configured as a device that provides continuous blood glucose monitoring and insulin injection and is configured to be worn on the patient's arm, as shown in FIG. 10. A block diagram for the device is shown in FIG. 11. The device is affixed to the patient by the attachment band 37 which uses a closure means such as a loop, button or Velcro (Velcro Inc., New Hampshire.) While the operation of the device is substantially automatic, controls 38 and display 39 are provided to interact with the device to obtain status information, turn the device off and on and to provide manual control of the device functions. Referencing FIG. 11, in addition to the needle 1, motor 2, motor controller 3 and power 4 of the previous implementation, the block diagram contains the following additional elements: display 39, controls (USERI) 38, pump 42, diagnostic sensor 40, and the separation of the motor function into separate motors, a long-travel, slow motor 43 and the insertion motor 44. The device uses a disposable cartridge containing the insulin reservoir 45, tube 46 and needle assembly 23 as shown in FIG. 13. The cartridge is installed in the device housing on the inner surface of the band prior to attaching to the patient. An interior view of the housing with the cartridge inserted is provided in FIG. 13. The insulin pump 42 function is provided, preferably, by a peristaltic pump whose motor 48 and screw 47 are shown in FIG. 12. The needle tip remains retracted in the cartridge until such time as the device is on the patient's arm and the START control is activated by the patient. On activating the START control, a preferably mechanical latch 49 releases a spring-loaded, viscous damped rotary arm 50 which then travels at a roughly linear velocity about its pivot point 51. At the end of the rotary arm is a pusher plate 52 with the piezoelectric insertion motor adhered to the side of the pusher plate 52 in contact with the needle assembly 23. During insertion of the cartridge into the device housing, mechanical features are provided on the cartridge and housing so as to retract the rotary arm and latch it into position. The needle assembly is predisposed to remain in the retracted position a bend in the tube 46 and the spring function which it provides as a result. At the time of the release of the rotary arm 50, the piezoelectric insertion motor is started and the needle is inserted into the patient's arm. In this implementation, the rotary arm provides the function of a long-travel, slow motor 43.

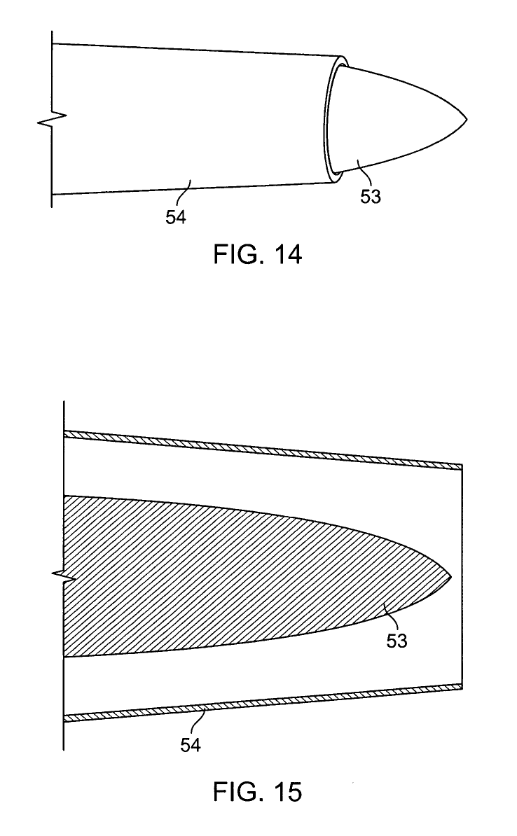

In one implementation, the needle assembly is composed of two elements providing the separate functions of diagnostic sensing and drug infusion. The diagnostic sensor for glucose measurement may take the form of a needle probe such as that described in U.S. Pat. No. 6,514,718 which uses standard amperometric sensing of glucose using a reagent such as glucose oxidase. Alternatively, the diagnostic sensing probe may be a fiber optic probe and the sensing means may be based on IR spectrometric methods for detection of glucose levels. In one implementation, the probe 1 providing the infusion function may be a hollow needle composed of a metal such as stainless steel or titanium of a diameter of preferably 200-300 μm, though diameters may be 10-3000 μm. Alternatively, the probe may be composed of a polymeric tube 54 such as polyurethane, polyolefin such as Engage (Dupont), Teflon (Dupont) or polyimide of the same diameter as shown in FIG. The polymeric tube will have an insertion needle 53 that is extended beyond the proximal tube of the polymeric tube 54 during insertion as shown in FIG. 14A, and then is retracted by the insertion motor when the motor is off or power is removed from the unit as shown in FIG. 15. The polymeric tube 54 is conical, i.e. its proximal end is of a narrower diameter than its distal end. When the insertion needle 53 is retracted, there is sufficient space between the surface of the insertion needle 53 and the inner wall of the polymeric tube 54 to allow for flow of the insulin. The polymeric tube may be composed of multiple materials arranged to provide a microporous region that allows for injection over a larger surface area than just the proximal tip of the tube.

In an alternative implementation, the pump 42 may be configured to allow both for insulin injection as well as removal of blood or other interstitial fluid for testing. The probe may also be configured with a cutting function either to provide a lancet function for drawing blood or for making very small incisions in membranes of various kinds. In some implementations, the cutting function is provided by serrations at the proximal end of the needle probe or along its length. In another implementation, the device provides only the glucose measurement function. This device is preferably inserted over one of the patient's fingers as shown in FIG. 16.

A great variety of implementations may be practiced. In some implementations, one or more of the following features may be incorporated. The motor element may be a piezoelectric actuator. The motor element may be a magnetic actuator. The magnetic actuator may incorporate a magnet affixed to the probe element with a coil element encircling the magnet/probe assembly. The motor element may be an electrostatic actuator. The motor power element may be a battery. The motor power element may be a mechanical source such as a spring or coil. A means may be provided for insertion of a flexible catheter substantially without the aid of a trocar, needle or guide wire. A flexible catheter whose flexural modulus differs substantially from its compressive modulus. A catheter whose proximal region is composed of a microporous material. A needle component of the probe that is hollow. A needle component of the probe made of metal, glass, or polymer. A needle component of the probe made of a carbon fullerene-based nanotube. A probe composed of a flexible optical material. An optical transceiver probe composed of an optical material composed of two or more fibers, one or more acting as transmitters, the remainder as receiver light guides. The optical transceiver probe with one or more of the transmitting fiber coated with an immobilized chemical reagent used for detection or measurement of a particular analyte. A wire or needle element, which may or may not be contained in the catheter lumen incorporating a biosensor for measurement of a body fluid constituent. The biosensor may incorporate a reagent for measuring glucose concentration. Some implementations may also include a pump element connected to the probe element for either withdrawing body fluids or infusing a fluid subcutaneously. The pump element may be comprised of a reservoir and piezoelectric pump mechanism. The probe element may be affixed to the device in such a way as to make the probe element disposable. The probe element assembly used for attaching the probe to the device housing may include a compliant element within the inner radius of the probe element assembly that annularly supports the probe but allows it to vibrate when actuated by the motor element. There may be more than one motor element, for instance the main motor providing small-scale higher frequency movements that reduces nociceptor activation and a longer travel, slower motor to insert the probe to extended depths. The probe element may include a force, compression or bend sensor such as a piezoelectric sensor for insertion feedback. The probe element may incorporate a cutting element to perform microsurgical operations or bloodletting in the form of a lancet. There may be more than one probe element, for instance one probe element that provides the biosensor function and another that provides a means of injecting a fluid. The device may be an attachment to existing manual hypodermic syringes. The velocity of the proximal end of the probe may be varied over time. The acceleration of the proximal end of the probe may be varied over time. The frequency of motion of the proximal end of the probe may be varied over time. The waveform describing the position of the proximal end of the probe may take the form of a monophasic rectilinear pulse. The waveform describing the position of the proximal end of the probe may take the form of a biphasic rectilinear pulse. The waveform describing the position of the proximal end of the probe may take the form of a sawtooth. The amplitudes of the pulses within the waveform pulse train may be randomized or semi-randomized.

Many other implementations of the invention other than those described above are within the invention, which is defined by the following claims.

1. A method for inserting at least one probe element through the skin to a penetration depth, the method comprising:
moving the probe element along a penetration path in a series of incremental movements,
the incremental movements producing incremental penetrations of the skin,
the incremental penetrations each being substantially smaller than the penetration depth, and
the incremental penetrations each being small enough not to produce substantial stimulation of nerve axons associated with nerve receptors along the penetration path.

2. A probe insertion device for assisting in inserting at least one probe element through the skin to a penetration depth, the device comprising:
probe movement elements for moving the probe element along a penetration path in a series of incremental movements,
the incremental movements producing incremental penetrations of the skin, the incremental penetrations each being substantially smaller than the penetration depth, and
the incremental penetrations each being small enough not to produce substantial stimulation of nerve axons associated with nerve receptors located along the penetration path.

3. The subject matter of claim 1 wherein the incremental penetrations are spaced apart in time to reduce stimulation of neurons along the penetration path.

4. The subject matter of claim 1 wherein moving the probe element along a penetration path comprises using probe movement elements.

5. The subject matter of claim 4 wherein the probe movement elements comprise: a probe element, a motor element, a motor control element, and a motor power element.

6. The subject matter of claim 5 wherein the probe element comprises one of a wire, a fiber, a hypodermic needle, a catheter with trocar.

7. The subject matter of claim 1 wherein there is a single probe element moved along the penetration path.

8. The subject matter of claim 4 wherein movement of the probe element is a combination of the effects of the probe movement elements and of manually applied force applied in the direction of penetration.

9. The subject matter of claim 4 wherein the probe movement elements produce an oscillatory movement of the probe element.

10. The subject matter of claim 4 wherein the probe movement elements produce a non-oscillatory movement of the probe element.

11. The subject matter of claim 1 wherein the majority of the incremental penetrations are between 1 μm and 1 mm.

12. The subject matter of claim 11 wherein the majority of the incremental penetrations are between 5 μm and 100 μm.

13. The subject matter of claim 9 wherein the oscillatory movement is primarily monophasic.

14. The subject matter of claim 9 wherein the oscillatory movement is primarily biphasic.

15. The subject matter of claim 9 wherein the oscillatory movement has a generally sawtooth waveform.

16. The subject matter of claim 15 wherein the rise time (insertion) of the individual sawtooth pulse is less than 1 ms.

17. The subject matter of claim 16 wherein the fall time (retraction) of the individual sawtooth pulse is greater than 1 ms.

18. The subject matter of claim 16 wherein the fall time (retraction) of the individual sawtooth pulse is greater than 20% of the pulse period.

19. The subject matter of claim 3 wherein the Insertion Pulse Spacing is greater than 50 μs.

20. The subject matter of claim 19 wherein Insertion Pulse Spacing is greater than 100 μs.

21. The subject matter of claim 20 wherein the Insertion Pulse Spacing is greater than 200 μs.

22. The subject matter of claim 13 wherein the majority of the oscillatory movements have Insertion Pulse Widths of between 10 μs and 10 ms.

23. The subject matter of claim 22 wherein a majority of the oscillatory movements have Insertion Pulse Widths of between 100 μs and 500 μs.

24. The subject matter of claim 1 wherein the slopes of the rising (insertion) edge and the falling (removal) edge of the pulse of the probe element is varied over time

25. The subject matter of claim 9 wherein the Insertion Pulse Width or Insertion Pulse Spacing of the oscillatory movement is varied over time.

26. The subject matter of claim 4 wherein the motor is a magnetic actuator.

27. The subject matter of claim 4 wherein the motor is a piezoelectric actuator.

28. The subject matter of claim 1 wherein movement of the probe element comprises an incremental movement superimposed on a gradual movement.

29. The subject matter of claim 28 wherein a short travel, incremental motor provides the incremental movement and a separate motive element provides the gradual movement.

30. The subject matter of claim 29 wherein the separate motive element comprises a spring providing inward pressure on the probe element along the direction of penetration.

31. The subject matter of claim 4 wherein the probe movement elements are configured to be attached to a manual hypodermic syringe.

32. The subject matter of claim 31 wherein the probe movement elements comprises one or more compliant elements that support the probe element but that allow it to vibrate when actuated by the motor element.

33. The subject matter of claim 32 wherein the probe element comprises a shaft suspended within a thread mount barrel by a flexible diaphragm.

34. The subject matter of claim 4 wherein the probe element comprises a flexible catheter made of a polymeric material.

35. The subject matter of claim 4 wherein the probe element comprises oriented polypropylene film.

36. The subject matter of claim 4 further comprising a pump element connected to the probe element for either withdrawing body fluids or infusing a fluid subcutaneously.

37. The subject matter of claim 36 wherein the pump element may be comprised of a reservoir and a piezoelectric pump mechanism.

38. The subject matter of claim 36 wherein the pump element comprises a piezoelectrically driven pump.

39. The subject matter of claim 36 wherein the pump element comprises a solenoid-based pump.

40. The subject matter of claim 36 wherein the pump is screw driven.

41. The subject matter of claim 4 wherein the probe element is affixed to at least some of the probe movement elements to make the probe element disposable.

42. The subject matter of claim 4 wherein the probe movement elements comprise a compliant element within the inner radius of a probe element assembly that annularly supports the probe but allows it to vibrate when actuated by the motor element.

43. The subject matter of claim 42 wherein the compliant element is pre-stressed in the retracted position allowing for faster activation during insertion.

44. The subject matter of claim 4 wherein the probe element comprises a needle component made of metal, glass, or polymer.

45. The subject matter of claim 44 wherein the needle component is made of a carbon fullerene-based nanotube.

46. The subject matter of claim 4 wherein there are more than one probe element undergoing the incremental penetration.

47. The subject matter of claim 44 wherein one probe element provides a biosensor function and another probe element provides a means of injecting a fluid.

48. The subject matter of claim 4 wherein the probe element includes a force, compression or bend sensor to provide insertion feedback.

49. The subject matter of claim 46 wherein the force, compression, or bend sensor comprises a piezoelectric sensor.

50. The subject matter of claim 4 wherein the probe element incorporates a cutting element to perform microsurgical operations or bloodletting in the form of a lancet.

51. The subject matter of claim 4 wherein the probe element comprises a flexible optical material.

52. The subject matter of claim 4 wherein the probe element comprises an optical transceiver probe comprised of an optical material composed of two or more fibers, one or more acting as transmitters, and the remainder as receiver light guides.

53. The subject matter of claim 50 wherein one or more of the transmitting fibers is coated with an immobilized chemical reagent used for detection or measurement of a particular analyte.

54. The subject matter of claim 4 wherein the probe element comprises a wire or needle element, which may or may not be contained in a catheter lumen incorporating a biosensor for measurement of a body fluid constituent.

55. The subject matter of claim 54 wherein the biosensor incorporates a reagent for measuring glucose concentration.

56. The subject matter of claim 1 wherein an additional motion is added that is orthogonal to the longitudinal axis of the probe element.

57. The subject matter of claim 1 wherein the nerve axons are those of nociceptors.
